(19) United States
(12) Patent Application Publication
(10) Pub. No.: US 2026/0157675 A1
(43) Pub. Date: Jun. 11, 2026

(54) DETECTION OF SHORT-CIRCUITED ELECTRODES IN MAPPING CATHETERS

(71) Applicant: BIOSENSE WEBSTER (ISRAEL) LTD., Yokneam (IL)

(72) Inventors: Yael Buchnik, Haifa (IL); Vadim Gliner, Haifa (IL)

(21) Appl. No.: 18/971,441

(22) Filed: Dec. 6, 2024

(51) Int. Cl. A61B 5/287; A61B 5/367

(52) U.S. Cl. CPC A61B 5/287; A61B 5/367; A61B 2560/0276

(57) ABSTRACT

A method includes receiving diagnostic electrical signals acquired over a given time window from multiple electrodes disposed over a distal end assembly of a catheter located inside a cardiac chamber. A level of similarity is determined between couples of the received diagnostic electrical signals. Electrical signals are identified as having a level of similarity that is indicative of a short circuit between the respective electrodes. A responsive action to the identification is initiated.

### FIELD OF THE DISCLOSURE

The present disclosure relates generally to invasive medical probes, and particularly to the detection of signals from short-circuited electrodes of the probe.

### BACKGROUND OF THE DISCLOSURE

Certain catheters used for cardiac electroanatomical mapping and electrically ablating cardiac tissue include multiple electrodes disposed over a distal end assembly of the catheter and electrically connected to a proximal end of the catheter. Multiple electrodes in a small space provide the catheter with precision and accuracy.

The present disclosure will be more fully understood from the following detailed description of the examples thereof, taken together with the drawings, in which:

### BRIEF DESCRIPTION OF THE DRAWINGS

FIG. 1 is a schematic, pictorial illustration of a catheter-based electroanatomical (EA) mapping and ablation system, according to an example of the present disclosure;

FIGS. 2A and 2B are schematic illustrations of sets of unipolar intracardiac electrograms (IEGM) that show the same signal on two electrodes of the catheter of FIG. 1, according to some examples of the present disclosure; and

FIG. 3 is a flow chart that schematically illustrates a method and algorithm to detect matching signals from short-circuited electrodes of the catheter of FIG. 1, according to an example of the present disclosure.

### DETAILED DESCRIPTION OF EXAMPLES

### OVERVIEW

Cardiac arrhythmias, such as atrial fibrillation and ventricle tachycardia, can be characterized using an electroanatomical (EA) mapping catheter containing multiple electrodes.

During EA mapping, electrical activity at regions in the heart is typically sensed and measured by advancing the catheter into the heart and acquiring multiple data points comprising a location and an electrophysiological value at the location (e.g., activation amplitude and/or timing). A processor generates an EA map from these data points to assist a physician to pinpoint electrophysiologically aberrant tissue areas. Subsequently, this and other arrhythmias can be treated by ablating the pinpointed arrhythmogenic regions in cardiac tissue.

For greater spatiotemporal resolution of EA mapping, it is desirable for the mapping catheter to conform closely to target anatomy, and to collect large amounts of data signals within a short time span. The catheter should be capable of allowing sufficient electrode contact with different tissue surfaces, such as flat, curved, irregular, or nonplanar surface tissue, and also be collapsible for atraumatic advancement and withdrawal through a patient’s vasculature.

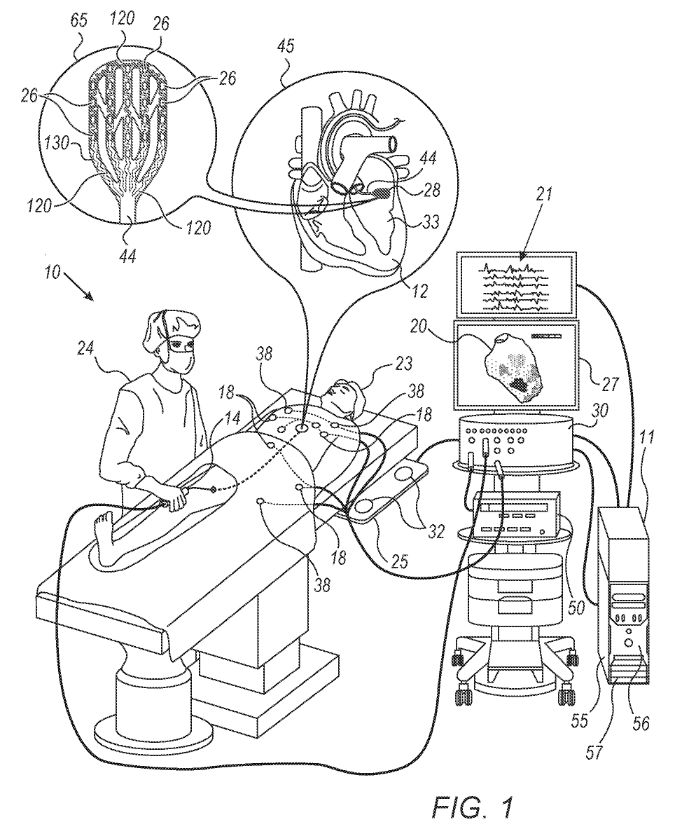

A flexible multi-electrode assembly (for example the planar catheter shown in FIG. 1) is suitable for efficient EA mapping of both large areas and curved portions of a cardiac chamber. The flexible flat assembly touches tissue gently to prevent invoking an arrhythmia during the EA mapping while readily conforming to cardiac chamber anatomy.

However, a flexible multi-electrode assembly is also prone to electrical shorts between conductive traces and/or electrodes, which result in the same EA signal on two electrodes. Such shorts may be temporary if, for example, the catheter folds too much as it conforms to a highly curved or compact anatomy.

If such electrical shorts go unnoticed and remain unmitigated, the same signal on two electrodes produces an erroneous data point that results in an incorrectly formed EA map.

Examples of the present disclosure described hereinafter provide a technique to identify very similar (e.g., almost identical) signal on two electrodes of the catheter (e.g., same unipolar intracardiac electrograms (IEGM)) during any given time window within the diagnostic session, such as EA mapping. Identifying electrical signals having a level of similarity is indicative of a short circuit between the respective electrodes. Following such identification, a processor of the system removes wrong data points, which were derived from the signals over this time window, from a data set used for generating the EA map.

In another example, the processor determines which electrodes are short-circuited during the time window, based on the identified signal identity, and may then determine that the distal end assembly is in a folded configuration during the time window.

The technique can monitor the electrical integrity of the mapping catheter in real time during EA mapping, or can be used during offline EA map generation. In one example, a processor performs cross-correlation between each electrical signal and all other electrical signals in the time window. If the processor finds a cross-correlation above a threshold value, the processor omits the data points acquired by the short-circuited electrodes at that time window to prevent damage to the EA map. The next time window may partially overlap with the previous one to ensure monitoring with a high temporal resolution.

The similarity threshold is set high enough so that correct signals that are similar will not be discarded.

In another example, the processor subtracts each electrical signal and all other electrical signals for each of the time windows. The processor calculates an average of the difference over the time window (to minimize impact of noise). If the processor finds the average difference to be below a threshold value, the processor omits the data points acquired by the short-circuited electrodes at that time window when generating the EA map. The threshold is set close enough to zero so that similar signals that are nevertheless correct will not be discarded.

### SYSTEM AND CATHETER DESCRIPTION

FIG. 1 is a schematic, pictorial illustration of a catheter-based electroanatomical (EA) mapping and ablation system 10, according to an example of the present disclosure. A physician 24 deploys a catheter 14 (e.g., flat or planar catheter, illustrated in inset 45) of system 10 in a chamber of a heart 12 (e.g., in a blood pool of a ventricle 33).

Specifically, a physician 24 advances the flat type of expandable distal-end assembly 28 (also called hereinafter “expandable distal-end assembly 28”) fitted on a shaft 44 of catheter 14 into contact with the heart wall for EA sensing a target site in heart 12.

As seen in inset 65, flat assembly 28 includes multiple functional electrodes 26 disposed over a nonconductive flexible substrate 130 (e.g., flexible printed board). Electrodes 26 may sense bipolar or unipolar IEGM signals with high spatial resolution. The electrodes are electrically connected using conductive traces 120 disposed over substrate 130 to a cable (not shown) running in shaft 44.

Distal-end assembly 28 may be constructed of multiple flexible non-conductive layers and flexible circuit layers. Electrodes 26 are disposed on both facets of assembly 28 to form a double-sided planar catheter.

Details of planar catheter 14 can be found in U.S. Provisional Patent Application S.N.63/406,673 (Attorney Docket No. BIO6749USPSP3) filed on September 14, 2022, and incorporated by reference in its entirety into this application as if outlined in full and attached in the Appendix to priority application U.S. Provisional Patent Application No. 63/505,764 (Attorney Docket No. 253757.000380 BIO6846USPSP1) filed June 02, 2023.

System 10 further includes one or more electrode patches 38 positioned for skin contact on patient 23 to enable (i) sensing unipolar IEGM signals (e.g., between an electrode 26 and a common electrode realized by one or more patches 38), and (ii) impedance-based tracking positions of functional electrodes 26.

For impedance-based position tracking, electrical current is directed toward electrode 26 and sensed at electrode skin patches 38, such that the location of each electrode can be triangulated via electrode patches 38. Details of the impedance-based location tracking technology are described in US Patent Nos. 7,536,218; 7,756,576; 7,848,787; 7,869,865; and 8,456,182.

A recorder 11 displays electrograms 21 captured with body surface ECG electrodes 18 and IEGM signals captured with functional electrodes 26 of catheter 14. Recorder 11 may include pacing capability for pacing the heart rhythm and/or may be electrically connected to a standalone pacer.

System 10 may include an ablation energy generator 50 adapted to conduct ablative energy to a subset of the plurality of electrodes 26 at the distal assembly 28 of catheter 14 configured for electrical ablation.

A patient interface unit (PIU) 30 is configured to establish electrical communication between catheter 14, electrophysiological equipment, power supply, and a workstation 55 for controlling the operation of system 10. Electrophysiological equipment of system 10 may include, for example, other catheters, location pad 25, body surface ECG electrodes 18, electrode patches 38, ablation energy generator 50, and recorder 11. Optionally, and preferably, PIU 30 additionally includes processing capability for implementing real-time computations of catheter locations and for performing ECG calculations.

Workstation 55 includes memory 57, a processor 56 with memory or storage with appropriate operating software loaded therein, and user interface capability. Workstation 55 may provide multiple functions, optionally including (i) modeling endocardial anatomy in three-dimensions (3D) and rendering the model or anatomical map 20 for display on a display device 27, (ii) displaying on display device 27 activation sequences (or other data) compiled from recorded electrograms 21 in representative visual indicia or imagery superimposed on the rendered anatomical map 20, (iii) displaying real-time location and orientation of multiple catheters within the heart chamber, and (iv) displaying sites of interest, such as places where ablation energy has been applied, on display device 27. One commercial product embodying elements of system 10 is available as the CARTO^{TM}3 System, available from Biosense Webster, Inc., 31A Technology Drive, Irvine, CA 92618.

### SIGNALS FROM SHORT-CIRCUITED ELECTRODES IN MAPPING CATHETERS

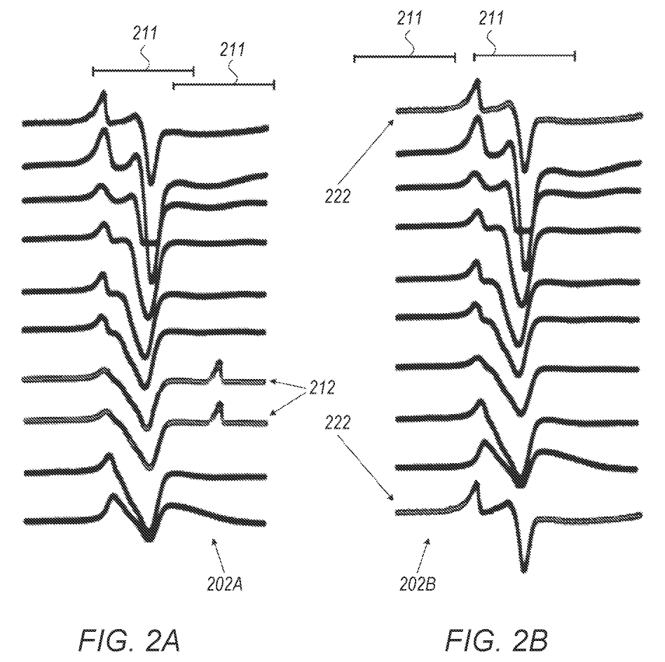

FIGS. 2A and 2B are schematic illustrations of sets 202A and 202B of IEGM signals that show the same signal on two electrodes 26 of catheter 14 of FIG. 1, according to some examples of the present disclosure.

Waveform sets 202A and 202B are acquired by multiple electrodes 26 of distal end assembly 28 in a cardiac chamber.

A processor, such as processor 56, may identify a level of similarity between each electrical signal and all other electrical signals within one or more time windows 211. The time windows 211 may partially overlap to ensure high temporal resolution monitoring. Specifically, the processor determines a level of similarity between couples of the received diagnostic electrical signals, going all over possible couples or a predefined set of couples (e.g., based on a set of electrodes 26).

In FIG. 2A, the two identical waveforms 212 are from adjacent short-circuited electrodes. In FIG. 2B, the two identical waveforms 222 are from non-adjacent short-circuited electrodes.

Depending on the electrical architecture (e.g., of the conductive circuits or traces), a similarity in waveform indicative of a short-circuit may therefore occur for any two electrodes 26 of the catheter, i.e., either physically adjacent or non-adjacent.

The waveforms may be identical for a brief duration, after which the temporary short circuit disappears, e.g., due to changed mechanical stress on the flexible catheter assembly 28.

It would be appreciated that the disclosed technique may also be applied to a situation in which multiple mapping catheters are located within the cardiac chamber, and electrodes from different catheters may come into contact with one another, causing a short circuit between them. In such a scenario, the disclosed technique could be applied for detecting shorted electrodes, each one of a different catheter.

### METHOD OF DETECTION OF SHORT-CIRCUITED ELECTRODES IN A MAPPING CATHETER

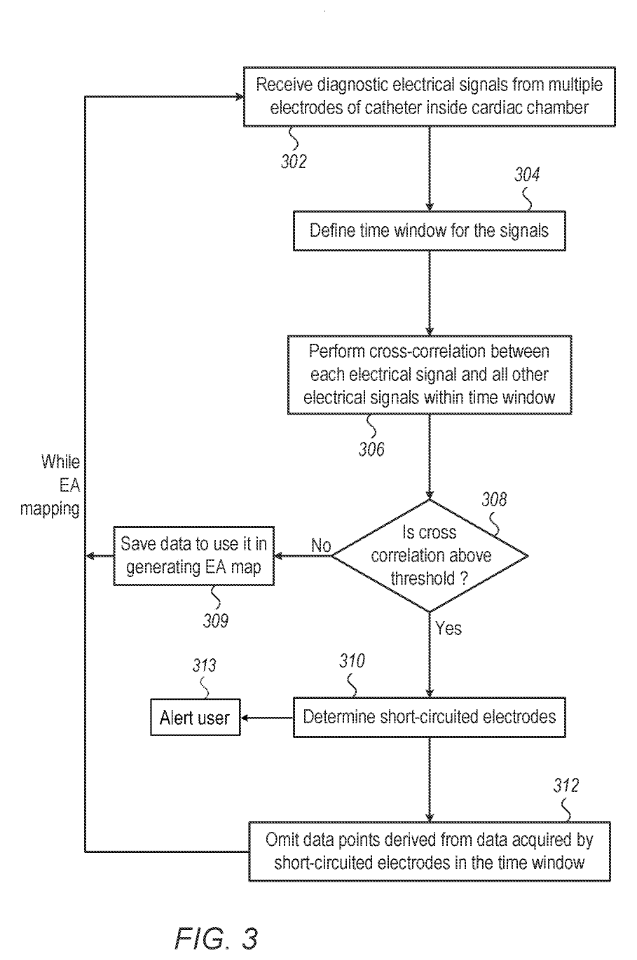

FIG. 3 is a flow chart that schematically illustrates a method and algorithm to detect matching signals from short-circuited electrodes 26 of catheter 14 of FIG. 1, according to an example of the present disclosure. The algorithm, according to the present example, carries out a process that begins at a signal receiving step 302, with processor 56 receiving diagnostic electrical signals 202 (unipolar IEGM signals) from multiple electrodes 26 disposed over distal end assembly 28 of catheter 14 inside a cardiac chamber.

At time windowing step 304, processor 56 defines a time window 211 over the signals received in step 302.

In signal similarity calculation step 306, the processor performs cross-correlation between each electrical signal and all other electrical signals in the time window.

If, in similarity checking step 308, the processor finds the cross-correlation above a threshold value, the processor identifies (310) the short-circuited electrodes and omits (312) the data points acquired by the short-circuited electrodes at that time window to prevent wrong data points from being included in the EA map. The processor alerts (313) the user about the specific shorted electrodes.

If, in similarity checking step 308, the processor finds the cross-correlation below a threshold value, the processor saves (309) the data points acquired and uses them in generating the EA map.

Optionally, for each step 308, the processor may check similarity over several time windows. A next time window may partially overlap the previous one to ensure high temporal resolution monitoring.

For the duration of the EA mapping session, the process returns to step 302 to receive a new set of waveforms.

The example flow chart shown in FIG. 3 is simplified for the sake of conceptual clarity. For example, additional steps can be considered, such as identifying short-circuited electrodes and using that information to determine if planar assembly 28 is in a folded configuration during the time window.

### EXAMPLES

### Example 1

A method includes receiving (302) diagnostic electrical signals (202) acquired over a given time window (211) from multiple electrodes (26) disposed over a distal end assembly (28) of a catheter (14) located inside a cardiac chamber (33). A level of similarity is determined between couples of the received diagnostic electrical signals. Electrical signals (212) are identified (310) as having a level of similarity that is indicative of a short circuit between the respective electrodes (26). A responsive action (312) to the identification is initiated.

### Example 2

The method according to example 1, wherein identifying (310) the level of similarity that is indicative of the short circuit comprises identifying that the level of similarity exceeds (308) a similarity threshold.

### Example 3

The method according to any of examples 1 and2, wherein receiving (302) the electrical signals comprises acquiring unipolar signals during the given time window (211).

### Example 4

The method according to any of examples 1 through 3, wherein identifying (310) the level of similarity comprises performing cross-correlation (306) between the electrical signals (202).

### Example 5

The method according to any of examples 1 through 3, wherein identifying (310) the level of similarity comprises calculating a difference between the electrical signals (202) and finding an average of the difference.

### Example 6

The method according to any of examples 1 through 5, wherein initiating the responsive action comprises omitting (312) data points derived from the identified (310) electrical signals (212) from a subsequent computation.

### Example 7

The method according to any of examples 1 through 6, wherein initiating the responsive action comprises notifying (313) a user of the short circuit.

### Example 8

The method according to any of examples 1 through 7, and comprising, based on identities of the electrodes (26) having the short circuit, detecting that the distal end assembly (28) is in a folded configuration during the time window (211).

### Example 9

The method according to any of examples 1 through 8, wherein the similarity threshold is set higher than the level of similarity between any correct but similar signals (202) of a data set of the received signals.

### Example 10

The method according to claim 1, wherein the distal end assembly (28) is a planar assembly.

### Example 11

A system (10) includes an interface (30) and a processor (56). The interface (30) is configured to receive diagnostic electrical signals (202) acquired over a given time window (211) from multiple electrodes (26) disposed over a distal end assembly (28) of a catheter (14) located inside a cardiac chamber (33). The processor (56) is configured to (i) determine a level of similarity between couples of the received diagnostic electrical signals (202), (ii) identify electrical signals (212) having a level of similarity that is indicative of a short circuit between the respective electrodes (26), and (iii) initiate a responsive action (312) to the identification.

Although the examples described herein mainly address cardiac diagnostic applications, the methods and systems described herein can also be used in other medical applications.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A method, comprising:
receiving diagnostic electrical signals acquired over a given time window from multiple electrodes disposed over a distal end assembly of a catheter located inside a cardiac chamber;
determining a level of similarity between couples of the received diagnostic electrical signals;
identifying electrical signals having a level of similarity that is indicative of a short circuit between the respective electrodes; and
initiating a responsive action to the identification.

2. The method according to claim 1, wherein identifying the level of similarity that is indicative of the short circuit comprises identifying that the level of similarity exceeds a similarity threshold.

3. The method according to claim 1, wherein receiving the electrical signals comprises acquiring unipolar signals during the given time window.

4. The method according to claim 1, wherein identifying the level of similarity comprises performing cross-correlation between the electrical signals.

5. The method according to claim 1, wherein identifying the level of similarity comprises calculating a difference between the electrical signals, and finding an average of the difference.

6. The method according to claim 1, wherein initiating the responsive action comprises omitting data points derived from the identified electrical signals from a subsequent computation.

7. The method according to claim 1, wherein initiating the responsive action comprises notifying a user of the short circuit.

8. The method according to claim 1, and comprising, based on identities of the electrodes having the short circuit, detecting that the distal end assembly is in a folded configuration during the time window.

9. The method according to claim 1, wherein the similarity threshold is set higher than the level of similarity between any correct but similar signals of a data set of the received signals.

10. The method according to claim 1, wherein the distal end assembly is a planar assembly.

11. A system, comprising:
an interface configured to receive diagnostic electrical signals acquired over a given time window from multiple electrodes disposed over a distal end assembly of a catheter located inside a cardiac chamber; and
a processor configured to:
determine a level of similarity between couples of the received diagnostic electrical signals;
identify electrical signals having a level of similarity that is indicative of a short circuit between the respective electrodes; and
initiate a responsive action to the identification.

12. The system according to claim 11, wherein the processor is configured to identify the level of similarity that is indicative of the short circuit by identifying that the level of similarity exceeds a similarity threshold.

13. The system according to claim 11, wherein the interface is configured to receive the electrical signals by acquiring unipolar signals during the given time window.

14. The system according to claim 11, wherein the processor is configured to identify the level of similarity by performing cross-correlation between the electrical signals.

15. The system according to claim 11, wherein the processor is configured to identify the level of similarity by calculating a difference between the electrical signals, and finding an average of the difference.

16. The system according to claim 11, wherein the processor is configured to initiate the responsive action by omitting data points derived from the identified electrical signals from a subsequent computation.

17. The system according to claim 11, wherein the processor is configured to initiate the responsive action by notifying a user of the short circuit.

18. The system according to claim 11, wherein the processor is further configured to, based on identities of the electrodes having the short circuit, detect that the distal end assembly is in a folded configuration during the time window.

19. The system according to claim 11, wherein the similarity threshold is set higher than the level of similarity between any correct but similar signals of a data set of the received signals.

20. The system according to claim 11, wherein the distal end assembly is a planar assembly.
